(19) United States
(12) Patent Application Publication
(10) Pub. No.: US 2026/0158111 A1
(43) Pub. Date: Jun. 11, 2026

(54) USE OF TGF IN THE PREVENTION VIRUS INFECTION OF THE RESPIRATORY TRACT

(71) Applicant: FrieslandCampina Nederland B.V., Amersfoort (NL)

(72) Inventors: Vassilios Triantis, Amsterdam (NL); Lamberta Hendrika Ulfman, Bunnik (NL); Ruprecht Jules Joost Van Neerven, Wageningen (NL)

(21) Appl. No.: 18/707,549

(22) PCT Filed: Nov. 4, 2022
(86) PCT No.: PCT/EP2022/080824

§ 371 Date: May 3, 2024

(30) Foreign Application Priority Data
Nov. 5, 2021 (EP) 21206692.2
Nov. 9, 2021 (EP) 21207244.1

(51) Int. Cl. A61K 38/18; A23L 33/00; A61K 35/20

(52) U.S. Cl. CPC A61K 38/1841; A23L 33/40; A61K 35/20

(57) ABSTRACT

A composition comprising Transforming Growth Factor beta 2 (TGF β2) for use in the prevention of viral infection of the respiratory tract.

### INTRODUCTION

This invention relates to compositions comprising transforming growth factor, in particular transforming growth factor beta for use in the prevention of an infection of the respiratory tract. The invention further relates to such compositions and the use thereof in preventing airway infections. The invention also relates to a method for the prevention of a virus infection of the respiratory tract.

### BACKGROUND

Breastfeeding is the best way to ensure healthy growth and development of infants during the first months of life. When mothers cannot or choose not to breastfeed, an alternative for breast milk is needed. High-quality foods for infants and young children are available that are safe and scientifically developed according to their nutritional needs. Still there is an ongoing need to further improve alternative solutions to breast milk. Such improvements may come for example from new compositions, composition preparation methods or administration procedures.

An infant's immune system is vulnerable to gastrointestinal and respiratory infections as it is not yet fully developed. Breast feeding has been shown to protect against such infections, since it contains bioactive components that can act against pathogens. However, when breastfeeding is not possible, milk-based infant formula is used as an alternative for infant feeding. “Trained immunity” or “innate immune training” was identified as a mechanism that confers immune memory to the mammalian innate immune system e.g. in the defense against respiratory infections.

When peripheral blood mononuclear cell (PBMC)-derived monocytes are pre-treated with bacille Calmette-Guerin (BCG) their responses upon re-stimulation with a range of toll-like receptor (TLR) ligands or heat-killed bacteria are significantly increased with higher production of tumor necrosis factor-α (TNF-α) and interleukin (IL)-6. As a result, most research on innate immune training focuses on measuring TNF-α and IL-6 as key cytokines affected. Dietary components, such as bovine Immunoglobulin G (blgG), have also the capacity to induce trained immunity in vitro.

It has been known that milk products contain growth factors which are present in very low concentrations in the milk product. They can be characterized by their isoelectric point, which is relatively high compared to other milk proteins, and their molecular weight. Transforming Growth Factor is used to describe two classes of polypeptide growth factors, TGFα and TGFβ.

Transforming Growth Factor beta (TGFβ, also referred to as TGF-β or TGF-beta) is a multifunctional protein found in all mammalian tissues. TGF-β are a group of polypeptides consisting five distinct or closely related members, B1 to 35, having considerable structural and biological homologies. TGF-β are homodimeric proteins. Different TGF-β have been implicated in the development, differentiation and growth of tissue and the control of immune system function and carcinogenesis. The amino acid homology between human and bovine TGF beta and in particular of TGF-β2 is very high, indicating a similar physiological effect of both forms. TGF-β can be isolated from natural sources (e.g. blood platelets), mammalian milk or colostrum, or can be produced by recombinant cells. For example, TGF-β can be extracted from milk products such as milk or whey (WO 9200994 and WO 9529933, EP 489884 or WO 2001 025276).

An example of a therapeutical use of TGF-β is the use of TGF-β for preparing a pharmaceutical composition for preventing damage of the intestinal mucosa as a result of chemotherapy or radiotherapy.

WO 2009134489 relates to a method for treating an existing viral respiratory infection in a subject, said method comprising: selecting a subject with a viral respiratory infection; providing a therapeutic agent that inhibits interferon-gamma (IFNy); and administering the therapeutic agent to the selected subject.

WO2007038870 relates to a method for obtaining a dairy fraction enriched in TGF-β that can be used in the treatment of inflammatory disorders.

An upper respiratory tract infection (URTI) is an illness caused by an acute infection, which involves the upper respiratory tract, including the nose, sinuses, pharynx, larynx or trachea above the vocal folds. This commonly includes nasal obstruction, sore throat, tonsillitis, pharyngitis, laryngitis, sinusitis, otitis media, and the common cold. Most infections are viral in nature, and in other instances, the cause is bacterial. URTIs can also be fungal or helminthic in origin, but these are less common. In 2015, 17.2 billion cases of URTIs are estimated to have occurred. As of 2014, they caused about 3,000 deaths, down from 4,000 in 1990.

Respiratory viral infection starts with entry of the virus in the airways epithelium and activation of viral receptors such as TLR3 and RIG-I. When the airways epithelial cells are infected, they initiate an immune response by secreting various cytokines such as TNFa and IL6 in order to clear the infection.

Treatment of URTI using antibiotics is not recommended, and no good evidence exists for or against the effectiveness of over-the-counter cough medications for reducing coughing. According to a Cochrane review, a single oral dose of nasal decongestant in the common cold is modestly effective for the short-term relief of congestion in adults; however, data on the use of decongestants in children are insufficient. Mucolytics such as acetylcysteine and carbocystine are widely prescribed for upper and lower respiratory tract infection without chronic broncho-pulmonary disease. However, in 2013 a Cochrane review reported their efficacy to be limited. (https://en.wikipedia.org/under wiki/Upper_respiratory_tract_infection).

Accordingly, there is a need for an alternative and/or additional treatment of respiratory tract infections, in particular for URTI; even more in particular of the buccal or oral cavity. Preferably such treatment prevents the onset of viral infections of the respiratory tract, in particular of the upper respiratory tract. Compositions used in such treatment should be safe and preferably without adverse effects.

The inventors surprisingly found that a composition comprising TGF prevents the onset of a viral infection of the respiratory tract and may solve one or more of the above-mentioned problems.

### SUMMARY OF THE INVENTION

Accordingly, in one aspect the invention relates to a composition comprising Transforming Growth Factor (TGF) for use in the prevention of viral infection of the respiratory tract.

In another aspect the invention relates to a composition comprising TGF, preferably TGF-β.

In yet another aspect the invention relates to the use of such a composition in the prevention of a viral infection of the respiratory tract, preferably the upper respiratory tract.

In still another aspect the invention relates to method for the prevention of a viral infection of the respiratory tract comprising the administration of a composition comprising Transforming Growth Factor (TGF) to a subject in need thereof and to the use of a composition comprising Transforming Growth Factor (TGF) in a method for the prevention of a virus infection of the respiratory tract in a subject in need thereof.

### Definitions

As used herein, a synthetic composition is a composition which is artificially prepared and is containing at least one compound that is produced ex vivo chemically and/or biologically and/or physically, e.g. by means of chemical reaction, enzymatic reaction or by a fractionation process. An example of such a fractionation process is a process wherein bovine milk is separated into different fractions like a fat and protein fraction. For the avoidance of doubt, a synthetic composition is not made in vivo by man or animal.

As used herein, the term “prevention” in relation to a given disease or disorder means preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

TGF is referring to “transforming growth factor”, preferably a milk-derived TGF. As used herein, the “respiratory tract” is defined as the subdivision of the respiratory system involved with the process of respiration in mammals. It is divided into the upper airways and lower airways. The upper airways or upper respiratory tract includes the nose and nasal passages, paranasal sinuses, the pharynx, oral cavity and the portion of the larynx above the vocal folds (cords). The lower airways or lower respiratory tract includes the portion of the larynx below the vocal folds, trachea, bronchi and bronchioles. The lungs are included in the lower respiratory tract and include the respiratory bronchioles, alveolar ducts, alveolar sacs, and alveoli.

“Respiratory tract infections” are defined as an illness caused by an infection of the respiratory tract, i.e. of the upper and/or lower respiratory tract.

As used herein, “upper respiratory tract infections” (URTI) are defined as an illness caused by an acute infection, which involves the upper respiratory tract, including the nose, mouth, sinuses, pharynx, larynx or trachea above the vocal folds.

Different TGF-β are synthesized as precursor molecules containing a pro-peptide region (which is also referred to as latency-associated peptide) in addition to the TGF-β homodimer and/or may form latent complexes with other proteins. TGF-beta are generally secreted in a biologically latent form. The latent form cannot interact with its receptor and is therefore biologically inert. Proteolysis yields two products that assemble into dimers. A dimer protein from the N-terminal region is called the latency-associated peptide (LAP), while a second dimer from the C-terminal portion of the precursor is called the mature TGF-β. This mature TGF-β is the active form of TGF (i.e. the C-terminal portion), it is the form that is capable of interacting with its receptor and has biological effect. The conversion of latent TGF beta from latent to active is extensively described by N. Khalil (N. Khalil; Microbes and Infection, 1, 1999, 1255-1263).

As used herein the “active form of TGF-β” refers to the form of TGF-β that is capable of interacting with its receptor and has biological effect without proteolysis being required. The latent form of TGF-β is the inactive form, i.e. not capable of interacting with its receptor and not providing a biological effect.

Unless otherwise specified, all amounts are per gram of dry weight of composition. Likewise, wt %, or percentages by weight, are per gram of dry weight of composition.

### BRIEF DESCRIPTION OF THE DRAWINGS

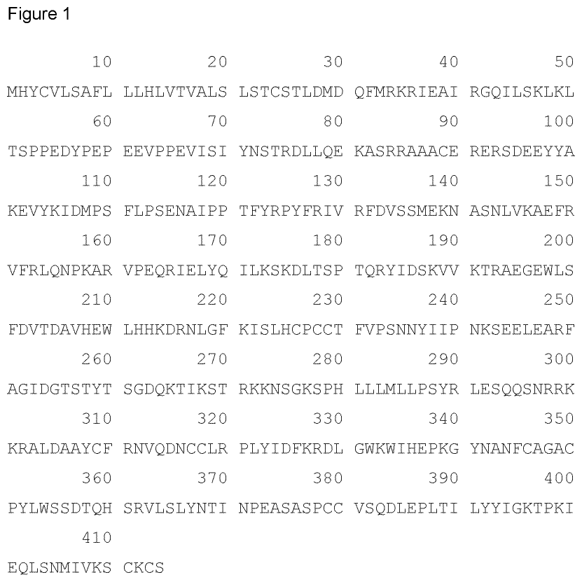

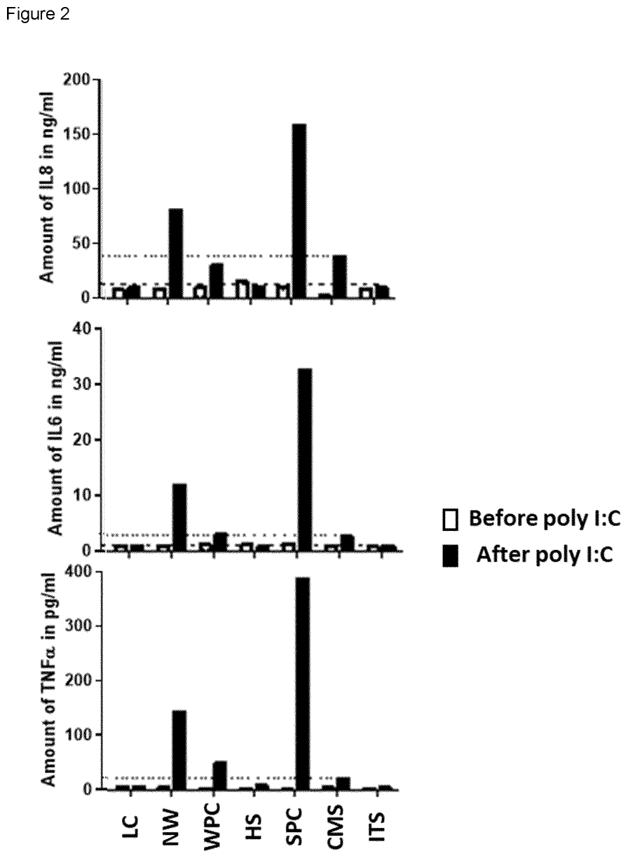

In FIG. 1 the amino acid sequence of bovine TGF beta 2 is shown. This is the amino acid sequence as defined in Uniprot under accession P21214 (https://www.uniprot.org/uniprot/P21214). Amino acid 1-20 (including) are herein defined as a signal peptide, amino acid 21-302 as the latency-associated peptide; and amino acid 303-414 as the active form of transforming growth factor beta-2. In FIGS. 2-7 the results of the experiments as performed in Examples 1 to 6 are shown.

The response from airway epithelial cells, pretreated with a milk fraction, after stimulation with a viral infection proxy (poly I:C) as shown by secreted levels of TNF-α, IL6 and IL8 is shown in FIG. 2.

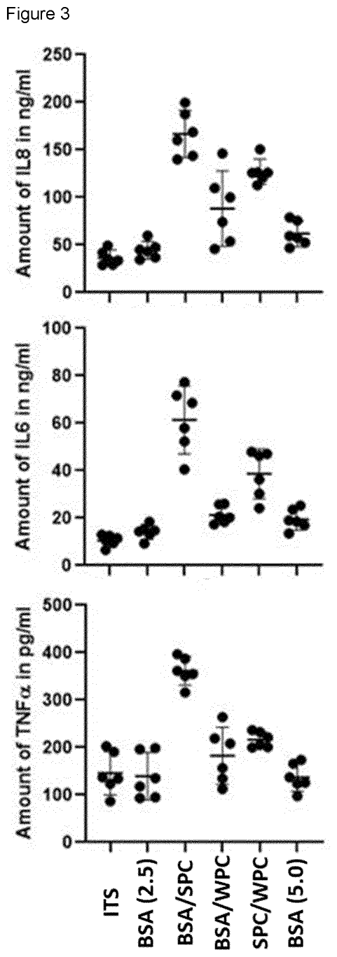

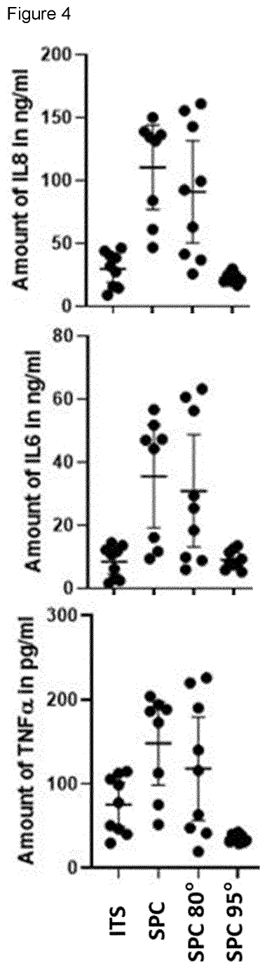

In FIG. 3 the response as shown by secreted levels of TNF-α, IL6 and IL8, from these cells is shown after pre-treatment with SPC, WPC or a combination thereof. The effect of mild and extensive heating of SPC on the ability of SPC to induce cytokine secretion after poly I:C stimulation is shown in FIG. 4.

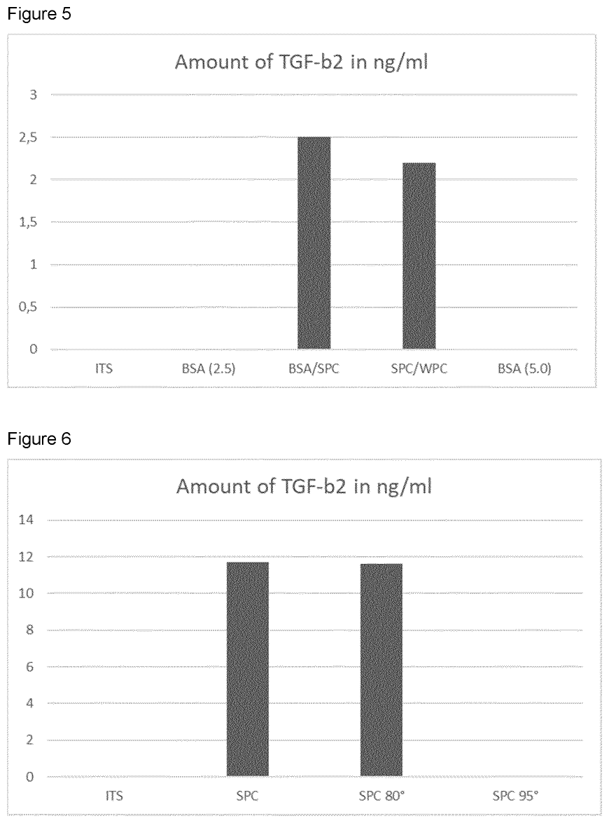

The levels of bovine TGF-β2 in the active milk fractions of examples 2 and 3 are shown in FIGS. 5 and 6, respectively.

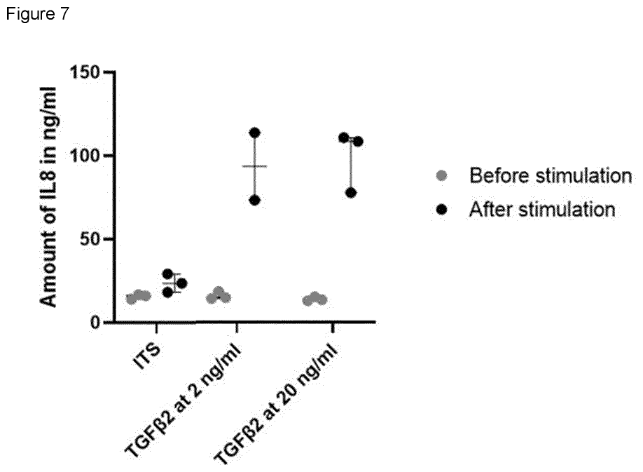

The response from airway epithelial cells, pretreated with bovine TGF-β2, after stimulation with a viral infection proxy (poly I:C) as shown by secreted levels of TNF-α, IL6 and IL8 is shown in FIG. 7.

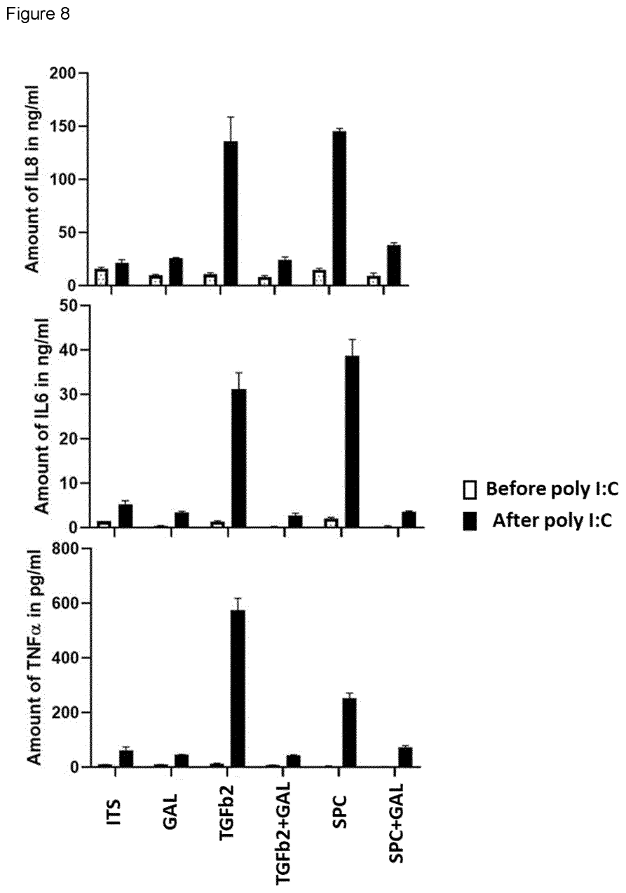

In FIG. 8 the results are shown of an experiment wherein cells incubated by 15 ng/ml of TGF-β2 (group TGFb2) or 5 mg/ml of SPC (total protein concentration, group SPC) showed enhanced cytokine production after poly I:C treatment compared to the control (group ITS). However when Galunisertib was added to TGF β or SPC (TGFb2+GAL, SPC+GAL, respectively) at a concentration of 100 nM then all activity was abolished, showing that TGF 32 is the only active ingredient in SPC since blocking specifically TGF β2 in SPC blocks SPC activity in total.

### DETAILS OF THE INVENTION

In one aspect the invention relates to a composition comprising Transforming Growth Factor (TGF) for use in the prevention of viral infection of the respiratory tract, preferably wherein the composition for use is a synthetic composition. Particularly, the invention relates to a synthetic composition comprising Transforming Growth Factor (TGF) for use in the prevention and/or preventing further development of viral infection of the respiratory tract, preferably for use in the prevention and/or further development of viral infection of the upper respiratory tract; wherein the TGF is TGF beta 2 (TGF 2). The inventors of the current invention surprisingly found that TGF is particularly effective in the prevention of viral infections of the upper respiratory tract. It is believed that mucosal tissue in the upper respiratory tract is in contact with TGF when a subject is drinking/consuming a composition comprising TGF. Without wishing to be bound by theory, it is believed that this contact triggers the subjects' immune system to provide more resistance towards viral infections. So, in one embodiment the composition comprising TGF is gargled for at least 2 seconds prior to swallowing it, preferably at least 5 seconds. So, in one embodiment the composition comprising TGF is gargled for 10 seconds, preferably 20 seconds, by the subject in need thereof to enhance the contact between the composition and the oral mucosal tissue. TGF β is particularly effective in the prevention of upper respiratory tract infections, especially TGF β2. So, in another embodiment the prevention of virus infection caused by the composition for use of the invention is a prevention of a virus infection of the upper respiratory tract. Preferably it is a prevention against sinusitis, common cold, pharyngitis.

The virus infection the composition of the invention prevents against, may be any virus infection that is known to infect the respiratory tract, in particular the upper respiratory tract, even more in particular the buccal cavity. The composition of the invention preferably prevents against a virus infection wherein the virus is selected from one or more of the group consisting of RSV, influenza virus, rhinoviruses, and adenoviruses; preferably wherein the virus infection is an infection with one or more of the group consisting of rhinoviruses, and adenoviruses; more preferably wherein the virus infection is a rhinoviruses.

Since bovine TGF 32 has a very high amino acid sequence identity when compared with human TGF β2, the TGF preferably is TGF from an mammalian origin, preferably from bovine, sheep, goat, horse, camel. Preferably, it is from a bovine origin, more preferably from a cow. TGF may be obtained from mammalian milk, so in one embodiment the TGF is milk-derived. Preferably, it is derived from mammal milk, more preferably bovine milk, sheep milk, goat milk, horse milk, camel milk, even more preferably bovine milk, most preferably cow's milk. The TGF may suitably be obtained from raw milk or milk protein-containing fractions. Examples of milk protein-containing fractions include whey protein concentrate (WPC), whey protein isolate (WPI), serum protein concentrate (SPC) or milk serum protein isolate (SPI). WPI, WPC, SPC, SPI are milk fractions well known in the art and readily commercially available. In one embodiment the TGF is as present in WPC i.e. WPC-derived; in one embodiment it is as present in WPI, i.e. WPI-derived; alternatively in another embodiment it is as present in SPC i.e. SPC-derived; alternatively in yet another embodiment it is as present in SPI i.e. SPI-derived. In still another embodiment the TGF is a mixture of WPC-derived and SPC-derived TGF, i.e. obtained from both WPC and SPC. Preferably, the TGF is SPC-derived.

Latent TGF beta may be activated by acid or heat treatment such as acid treatment in the stomach or heat treatment during pasteurization, or mild proteolysis. Accordingly, in one embodiment the TGF in the composition of the invention is in its active form; in one preferred embodiment the TGF-β in the composition of the invention is in its active form, more preferably the TGF-β is TGF-β2.

In another embodiment the amino acid sequence of TGF-β in the composition of the invention has more than 97% amino acid sequence identity (differs in less than 3 amino acids (i.e. insertions, deletions, and/or substitution) with the amino acid sequence of position 303-414 as shown in FIG. 1, which corresponds to the sequence as defined under ACCESSION P21214 in Uniprot (https://www.uniprot.org/uniprot/P21214).

“Sequence identity” and “sequence similarity” can be determined by alignment of two peptide or two nucleotide sequences using global or local alignment algorithms. Sequences may then be referred to as “substantially identical” or “essentially similar” when they are optimally aligned by for example the programs GAP or BE ST FIT or the Emboss program “Needle” (using default parameters, see below) and share at least a certain minimal percentage of sequence identity (as defined further below). These programs use the Needleman and Wunsch global alignment algorithm to align two sequences over their entire length, maximizing the number of matches and minimizes the number of gaps. Generally, the default parameters are used, with a gap creation penalty=10 and gap extension penalty=0.5 (both for nucleotide and protein alignments). For nucleotides the default scoring matrix used is DNAFULL and for proteins the default scoring matrix is Blosum62. Sequence alignments and scores for percentage sequence identity may for example be determined using computer programs, such as EMBOSS (http://www.ebi.ac.uk/rools/psa/emboss_needle/). Alternatively sequence similarity or identity may be determined by searching against databases such as FAST A, BLAST, etc., but hits should be retrieved and aligned pairwise to compare sequence identity.

In yet another embodiment the amino acid sequence of TGF-β in the composition of the invention differs in less than 3 amino acids with the active form of human TGF beta 2. In other words, when the TGF-β is aligned with human TGF-β2, there are less than 3 differences in the amino acid sequences i.e. insertions, deletions, and/or substitutions. Preferably, less than 2 differences, more preferably less than 1, most preferably the sequence is identical.

### Compositions Comprising TGF

The composition for use of the current invention may further comprise one or more of a carbohydrate fraction, a protein fraction and a lipid fraction; preferably wherein the composition comprises a carbohydrate fraction, a protein fraction and a lipid fraction. The composition for use of the invention preferably comprises a clinically effective amount of TGF, more preferably a clinically effective amount of TGF beta. As used herein, “clinically effective amount” means an amount of TGF that is at least 0.001 micro gram per gram of composition (i.e. per gram of ready to drink composition when the composition is a liquid; or alternatively per gram of dry weight of the composition if it is a dry/powdered composition). In one embodiment the amount of TGF is at least 10⁻⁷ wt % of TGF per gram of liquid (e.g. ready to drink) composition. Preferably the amount of TGF is at least 3*10⁻⁷ wt % per gram of ready to drink composition, more preferably, at least 10⁻⁶ wt %, even more preferably at least 10⁻⁵ wt %. In another embodiment the amount of TGF is at least 10⁻⁵ wt % referred to the total amount of protein in the composition, such as at least 2*10⁻⁵ wt %, 4*10⁻⁵ wt %, 6*10⁻⁵ wt % or 8*10⁻⁵ wt %. Even more preferably the amount of TGF is at least 10⁻⁴ wt % as referred to the total amount of protein. In still another embodiment the amount of TGF, such as the amount of TGF-β, is between 1 and 100 microgram per gram of composition. Preferably between 6 and 60 microgram per gram of composition, more preferably between 10 and 40, even more preferably between 10 and 20 microgram per gram of dry weight of composition. In a so-called high dosage form the amount of TGF, such as TGF beta, in the composition of the invention is at least 0.001 wt %, preferably 0.01 wt %, even more preferably 0.1 wt %. Normally the amount of TGF is below 5 wt %. In another embodiment, the composition according to the invention comprising TGF is a liquid, and the amount of TGF, such as the amount of TGF beta, is more than 0.01 nanogram per ml, preferably more than 0.1 ng/ml, even more preferably more than 1 ng/ml, particularly preferably more than 10 ng/ml. The maximum amount of TGF in the composition of the invention preferably is less than 100 microgram/ml (<100 μg/ml) such as less than 10 μg/ml or even less than 1 μg/ml. In yet another embodiment, the amount of TGF is between 1 nanogram per ml and 100 nanogram per ml of product, preferably between 2 and 75 nanogram per ml, more preferably between 10 and 50 nanogram per ml. It is understood that an amount per ml also relates to powdered compositions according to the invention that can be reconstituted with a liquid such as water. In yet another embodiment, the amount of TGF in the composition of the invention is at least 0.5*10⁻⁵ g per gram of protein, preferably at least 1*10⁻⁵ g per gram of protein more preferably at least 5*10⁻⁵ g per gram of protein. In still another embodiment, the amount of TGF in the composition is between 1*10⁻⁵ g and 100*10⁻⁵ g per gram of protein, preferably between 1*10⁻⁵ g and 10*10⁻⁵ g per gram of protein. Alternatively, in one embodiment the amount of TGF in the composition of the invention is at least 0.1*10⁻⁸ wt % of TGF per gram of dry weight of composition, preferably at least 0.1*10⁻⁸ wt %, more preferably between 0.1*10⁻⁸ wt % and 10*10⁻⁸ wt %. In an even more preferred embodiment, these amounts are the amounts of TGF-β2.

As is commonly known, viral infections of the respiratory tract may occur in people of all ages, young and old. Severity of such an infection may depend on the state of a subject's immune system. Subjects with an immune system which is not yet fully developed or a weakened immune system may be more susceptible towards viral infections of the respiratory tract. Accordingly, in one embodiment the composition for use of the current invention is an infant formula. Infant formulae are well known in the art.

The term “infant formula” as used herein refers to a nutritional composition intended for infants and as defined in Codex Alimentarius, (Codex STAN 72-1981) and Infant Specialties (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2 (c) of the European Commission Directive 2006/141/EC of 22 Dec. 2006 on infant formulae and follow-on formulae). The infant formulas can encompass the starter infant formulas, the follow-up or follow-on formulas, and young child formulas. Generally a starter formula is for infants from birth as breast-milk substitute, and a follow-up or follow-on formula from the 6th month onwards. So, an infant formula may be dedicated for infants of 0 to 6 months, 6 to 12 months, 12 months an older.

In another embodiment, the composition for use of the current invention is an adult nutrition product or in yet another embodiment a food supplement. The adult nutrition product can for example be a milk-based product such as a yoghurt drink, a protein bar, or other product. Compositions and forms of adult nutrition products are well known in the art and all variants are suitable as long as they allow the addition of a protein fraction comprising TGF. Preferably the adult nutrition product comprises milk protein, more preferably an TGF beta-comprising fraction of milk protein, such as SPC. Typical examples of suitable adult nutrition products include protein shots comprising protein (15-25 wt %), carbohydrate, fats and optional ingredients such as minerals and vitamins.

TGF or a TGF-comprising fraction may be added to an existing product to make it suitable for use against viral infections of the respiratory tract. Alternatively, proteinaceous material in an existing adult nutrition product may be replaced with milk protein comprising TGF.

The carbohydrate fraction in the composition for use of the current invention may comprise digestible and non-digestible saccharides. Digestible saccharides include glucose, galactose, sucrose, maltose, dextrin, and lactose. Preferably the composition of the invention comprises one or more non-digestible oligosaccharides, more preferably the non-digestible oligosaccharide is selected from the group consisting of fructo-oligosaccharides, galacto-oligosaccharides, arabino-oligosaccharides, arabinogalacto-oligosaccharides, glucooligosaccharides, chito-oligosaccharides, glucomanno-oligosaccharides, galactomannooligosaccharides, mannan-oligosaccharides, fuco-oligosaccharides, sialyl-oligosaccharides and N acetylglucosamine oligosaccharides; more preferably wherein the non-digestible oligosaccharide is selected from the group consisting of fructo-oligosaccharides and galacto-oligosaccharides. As used herein, fructo-oligosaccharides include inulin. Even more preferably wherein the non-digestible oligosaccharide is galacto-oligosaccharide. Particularly preferably wherein the non-digestible oligosaccharide is fructo-oligosaccharide. All such non-digestible oligosaccharides are readily commercially available.

In still another embodiment the carbohydrate fraction in the composition for use of the current invention comprises one or more human milk oligosaccharides (HMOs), as these are known for their various health effects. Preferably the one or more HMOs are selected from the group consisting of 2′FL, 3FL, 3′GL, 6′GL, 3′SL, 6′SL, LNnT, or LNT; or combinations thereof. As used herein, 2′FL is referring to 2′ Fucosyl lactose, 3FL to 3 Fucosyl lactose, 3′GL to 3′ galactosyl-lactose, 6′GL to 6′ galactosyl-lactose, 3′SL to 3′ sialyl lactose, 6′SL to 6′ sialyl lactose, LNnT to Lacto-N-neotetraose, LNT to Lacto-N-tetraose. Preferably, the total amount of HMOs is at least 0.1 wt %, more preferably at least 0.5 wt %. The total amount is preferably less than 10 wt %, more preferably less than 5 wt %.

Fat consists of a glyceride residue linked to a carboxylic acid via an ester bond. As such, a fat molecule comprises a glyceride residue and three fatty acid acyl groups. A acyl group linked to the first carbon of a glyceride is also referred to as an sn1 linked acyl group, like an acyl group linked to the second carbon of a glyceride is referred to as an sn2 linked acyl group and an acyl group linked to the third carbon of a glyceride is referred to as sn3 linked acyl group.

An acyl group contains a double-bonded oxygen atom and an alkyl group (R—C═O). In organic chemistry, the acyl group (IUPAC name: alkanoyl) is usually derived from a carboxylic acid. Therefore, it has the formula RCO—, where R represents an alkyl group that is linked to the carbon atom of the group by a single bond.

The lipid fraction in the composition for use of the current invention may comprise a mixture of different fats and oils, such as a mixture of plant oils and milk fat. Optionally long chain poly unsaturated fatty acids (LC-PUFA) such as DHA, ARA, EPA may be added to the lipid fraction. In one embodiment the lipid fraction has a fatty acid composition wherein at least 0.6 wt % of the fatty acid acyl groups consists of 4 carbon atoms, preferably at least 1.2 wt %, more preferably at least 1.5 wt %; and/or wherein at least 10% of the fatty acid acyl groups present in the lipid fraction is a palmitoyl group (CH₃(CH₂)₁₄C(O)) and at least 30% of the palmitoyl groups is esterified to the sn-2 position of a triglyceride based on total palmitoyl groups.

Plant oils for human consumption are well known in the art. Suitable milk fat for the composition of the invention is mammalian milk fat, preferably from a ruminant, more preferably from cow's milk, sheep milk, or goat milk, most preferably from cow's milk. For example whole milk, cream, butter, anhydrous milk fat and the like.

In one embodiment the amount of milk fat in the lipid fraction of the composition of the invention is at least 20 wt % of the lipid fraction, preferably at least 30 wt %, more preferably at least 40 wt %, even more preferably at least 60 wt %.

The protein fraction in the composition for use of the current invention may comprise other proteins in addition to TGF. For example plant proteins or other milk proteins such as whey proteins or casein.

In another aspect, the invention relates to a method for the prevention of a virus infection of the respiratory tract comprising the administration of a composition comprising Transforming Growth Factor (TGF) as defined elsewhere herein to a subject in need thereof. Preferably wherein the composition is a synthetic composition, more preferably, wherein the TGF is TGF-β2 even more preferably TGF-β2 in its active form. In one embodiment, the invention relates to a method for the prevention of a virus infection of the respiratory tract, preferably in the further development of a virus infection of the respiratory tract, comprising the administration of a synthetic composition as defined elsewhere herein to a subject in need thereof

In still another aspect, the invention relates to the use of a composition comprising Transforming Growth Factor (TGF) in the prevention of a virus infection of the respiratory tract, wherein the composition is as defined elsewhere herein; preferably, wherein the use comprises the step of administering the composition comprising TGF to a subject in need thereof. Preferably, the invention relates to the of a composition comprising TGF β2, in the prevention of a virus infection of the respiratory tract, preferably in the further development of a virus infection of the respiratory tract, wherein the composition is as defined herein; wherein the use comprises the step of administering the composition comprising TGF 32 to a subject in need thereof.

In yet another aspect, the invention relates to a composition as defined herein above wherein the composition comprises at least 20 nanogram TGF per ml; and/or at least 1*10⁻⁵ ng TGF per gram of protein; preferably wherein the TGF is TGF beta, more preferably wherein the TGF beta is in its active form. In one embodiment the amount of TGF is at least 50 ng per ml or even at least 100 ng per ml. In another embodiment the amount of TGF is at least 5*10⁻⁵ ng TGF per gram of protein, preferably at least 10*10⁻⁵ ng TGF per gram of protein.

It is to be understood that this invention is not limited to the specific embodiments and methods described herein, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a”, “an,” and “the” comprise plural referents unless the context clearly indicates otherwise. For example, reference to a component in the singular is intended to comprise a plurality of components.

Unless defined otherwise, all technical and scientific terms used herein generally have the same meaning as commonly understood by one of ordinary skill in the art to which this invention belongs.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies, mutatis mutandis, to normal grammatical variations of the initially defined abbreviation; and, unless expressly stated to the contrary, measurement of a property is determined by the same technique as previously or later referenced for the same property.

The invention is hereinafter illustrated with reference to the following, non-limiting, examples.

### EXAMPLES

Detroit D562 cells (ATCC-CCL138) were cultured in a T75 flask (Corning cat. No. 430641) in minimum essential medium (MEM, Invitrogen Catalog No. 21090-022) with 1× non-essential amino acids (Invitrogen cat. No. 11140-035), 10 mM sodium pyruvate (Invitrogen cat. No. 11360-039), 2 mM glutamax (Invitrogen cat. No. 35050-038), 1000 U/ml antibiotics penicillin, 1000 μg/ml streptavidin and 10% serum (Hyclone, GE healthcare Life Sciences, lot. No. RB35936), henceforth called 10% complete medium with serum (CMS).

Cells were trypsinized when confluency of 80% or greater was achieved, using 3 ml of Trypsin-EDTA (Invitrogen, cat. No. 25200-056) and resuspended into 12 ml of 10% CMS. Cells were counted and seeded at 6*10⁴ cells/ml in three 24-well tissue culture treated plate (Corning cat. No. 3527) with 1 ml per well. These were cultured for one week till confluent.

48 hours before the components were added, all cells were put on to 1% ITS (1 mg/ml Insulin, 0.55 mg/ml transferrin and 0.00067 mg/ml Selenium) to allow the cells to adjust to serum free conditions, as serum may contain immune active components that can mask the effects of the test ingredients. The only exception being the CMS control as this was also not be exposed to ITS in the incubation phase.

All the components were calibrated on protein concentration and added on cells at the calculated total protein concertation of 1 mg/ml on dry matter.

After 3 days supernatants were collected and the cells were washed and exposed to 10 μg/ml of poly I:C (i.e. Polyinosinic-polycytidylic acid). Poly I:C was dissolved in 1% ITS.

After 24 hours supernatant was collected and stored at −20° C. until IL-8, IL-6 and

TNF-α ELISAs could be performed. ELISA kits from R&D Systems (Biotechne) were used to quantify cytokines IL-8/CXCL8 (DY208), IL-6 (DY206), TNF-α (DY210) and IFN-λ1/3 (DY1598B) according to the manufacturer's guidelines. Absorbance was measured at 450 nm and corrected for at 570 nm using the Biorad iMark plate reader & Imark software version 6.2. Cytokines were quantified before and after the addition of poly I:C.

The following components were tested:

| | |
|---|---|
| Lactose control (LAC) | (from FrieslandCampina) |
| Native Whey (NW) | (from FrieslandCampina) |
| Whey protein concentrate (WPC) | (from FrieslandCampina) |
| Human serum (HS) | |
| Serum protein concentrate (SPC) | (from FrieslandCampina) |
| CMS (complete medium with serum) | |

All added components were tested for putative bacterial endotoxin contamination and were found negative. Native whey is whey with a high degree of protein nativity and may be obtained as described in e.g. WO2020159372 or EP3042565.

### Example 1

As shown in FIG. 2, WPC, Native Whey (NW) and Serum Protein Concentrate (SPC) induced a stronger response from airway epithelial cells after stimulation with a viral infection proxy (after poly I:C) than the control, as shown by secreted levels of TNF-α, IL6 and IL8. Cytokine secretion was not affected by the ingredients themselves before poly I:C stimulation. All samples were added at a final protein concentration of 1 mg/ml (calibrated on dry matter).

### Example 2

In this example it was tested whether addition of WPC to SPC could affect is activity. Cells were incubated with SPC or WPC alone at a total protein concentration of 2.5 mg/ml. A mixture of SPC and WPC was also tested (both streams were added at a concentration of 2.5 mg/ml having a final total protein concentration of 5 mg/ml (group SPC/WPC). In order to calibrate all groups to equal amounts of protein, BSA (2.5 mg/ml) was added to the groups of SPC (BSA/SPC) or WPC (BSA/WPC) so that the total amount of protein on the cells was equal in all treatments (5 mg/ml of total protein).

BSA at 2.5 mg/ml and 5 mg/ml was taken along as a control (BSA 2.5 and BSA 5.0 respectively). Cytokine levels in the supernatant of the cell cultures were measured 24 hours after poly I:C incubations.

As shown in FIG. 3, cells treated with SPC (BSA/SPC) and the combination (SPC/WPC) were more active than those treated with BSA (control) [statistically significant]. Treatment with SPC or the combination (SPC/WPC) resulted in larger amounts of IL8, IL6 and TNFα being secreted, which is indicative for a higher resistance against viral infections. Treatment with WPC (BSA/WPC) resulted in higher amounts of IL8 and TNFα being secreted as compared to the control.

### Example 3

In order to determine if it was a protein component in SPC that is responsible for the observed effect, SPC (at a total protein concentration of 5 mg/ml) was heated mildly (80° for 1 min) or extensively (95° for 10 min) in order to destroy thermosensitive proteins.

As shown in FIG. 4, extensive heating of SPC at 95° for 10 min abolished its activity showing that a protein component is responsible for its activity. This component was resistant to mild heat treatments (80° for 1 min) since such procedures retained the ability of SPC to induce higher cytokine secretion after poly I:C stimulation.

### Example 4

Since bovine TGF-β2 is a heat resistant protein that can be found in bovine milk it was concluded that bovine TGF was the compound responsible for the results. Hence in a further experiment the levels of bovine TGF-β2 were measured in the streams that were tested in EXAMPLE 2. Streams containing SPC or SPC/WPC showed measurable levels of bovine TGF-β2, as shown in FIG. 5.

### Example 5

The levels of bovine TGF-β2 were measured in the streams that were tested in EXAMPLE 3. Only streams that were active in the experimental set up of EXAMPLE 3 showed measurable levels of bovine TGF-β2, as shown in FIG. 6.

### Example 6

In this example it was tested whether bovine TGF-β2 alone could give similar results as the milk streams that we could measure it in. Therefore we tested in the same experimental set up bovine TGF-β2 at levels similar to those measured at EXAMPLES 4 and 5. Bovine TGF-β2 alone could induce airways epithelial cell training similarly to SPC in the previous examples, as shown in FIG. 7. Before stimulation with either 2 ng/ml or 20 ng/ml TGF-β2, the amount of IL8 was similar to the control (ITS). Cells incubated with TGF-β2, prior to stimulation with a viral infection proxy (poly I:C) produced significantly higher amounts of IL8 than the control, i.e. about 4 times higher.

### Example 7

In this example it was tested whether bovine TGF-β2 is the only active ingredient in SPC by using Galunisertib; a potent inhibitor of the TGF-β cellular receptor. As shown in FIG. 8, cells incubated by 15 ng/ml of TGF-β2 (group TGFb2) or 5 mg/ml of SPC (total protein concentration, group SPC) showed enhanced cytokine production after poly I:C treatment compared to the control (group ITS). However when Galunisertib was added to TGF β or SPC (TGFb2+GAL, SPC+GAL, respectively) at a concentration of 100 nM then all activity was abolished, showing that TGF 32 is the only active ingredient in SPC since blocking specifically TGF β2 in SPC blocks SPC activity in total. Galunisertib alone at the concentration of 100 nM had no effect on the cells.

### Example 8

An example of a nutritional composition (e.g. an infant formula) comprising bovine TGF β (from bovine milk) of the present invention is given in the below table 1.

TABLE 1

| | | Infant | Growing-Up |
|---|---|---|---|
| Ingredient | | Formula | milk |
| Skimmed milk (liquid) | kg solids/100 kg | 3-4 | 14-18 |
| milk protein concentrate | kg solids/100 kg | 14-16 | 17-21 |
| (SPC) | | | |
| cream | kg solids/100 kg | 14-16 | 9-12 |
| Lactose | kg/100 kg | 40-44 | 33-35 |
| vegetable fatblend | kg/100 kg | 12-15 | 8-12 |
| oil/minerals/vitamins | kg/100 kg | 2-4 | 1-3 |
| Galacto-oligosaccharides | kg/100 kg | 4-5 | 4-6 |
| nucleotides, trace elements | kg/100 kg | 1.1-1.9 | 0.5-2 |
| Whey Protein Concentrate | kg/100 kg | 1.5-5 | 2-4 |
| Lactoferrin | kg/100 kg | 0.1-0.5 | 0.1-0.5 |
| HMO | kg/100 kg | 0.1-0.5 | 0.1-0.6 |

### Example 9

An example of a nutritional composition (e.g. an adult nutrition product) comprising bovine TGF β (from bovine milk) of the present invention is given below. Ingredients: Water, milk protein (SPC), glucose syrup, vegetable oils, (sunflower, rapeseed), sucrose, fiber (fructooligosaccharides, inulin, galacto-oligosaccharides), minerals, fish oil, emulsifier, vitamins, carnitine, stabilizer, flavor, taurine. The composition comprises per serving size of 100.0 ml:

| Energy: | 550-800 | kJ |
|---|---|---|
| Protein: | 5-15 g (from SPC), | |
| Dietary Fiber: | 1-5 | g |
| Fat: | 5-10 | g |
| Carbohydrate: | 10-15 | g |
| Alpha Linolenic Acid: | 150-200 | mg, |
| Calcium: | 200-350 | mg |
| Poly Un Saturated Fat: | 3-5 | g. |

1. A synthetic composition comprising Transforming Growth Factor (TGF) for use in the prevention and/or preventing further development of viral infection of the respiratory tract, preferably for use in the prevention and/or further development of viral infection of the upper respiratory tract; wherein the TGF is TGF beta 2 (TGF β2).

2. The synthetic composition of claim 1 for use in preventing the further development of viral infection in the upper respiratory tract.

3. The synthetic composition for of claim 1, wherein the TGF β2 is milk-derived TGF β2; preferably derived from mammal milk, more preferably bovine milk, sheep milk, goat milk, horse milk, camel milk, even more preferably bovine milk, most preferably cow's milk.

4. The synthetic composition of claim 1, wherein the milk-derived TGF β2 is whey protein concentrate-derived (WPC-derived) or serum protein derived (SPC-derived), preferably wherein the milk-derived TGF is SPC-derived.

5. The synthetic composition of claim 1, wherein the composition further comprises one or more of a carbohydrate fraction, a protein fraction and a lipid fraction, preferably wherein the composition comprises a carbohydrate fraction, a protein fraction and a lipid fraction.

6. The synthetic composition of claim 1, wherein the amount of TGF β2 is one or more of:
i. at least 10⁻⁷ wt % referred of the liquid composition;
ii. at least 0.5*10⁻⁵ g per gram of protein in the composition; and/or
iii. at least 0.01 nanogram per 1 ml of composition, preferably at least 1 nanogram/ml.

7. The synthetic composition of claim 1, wherein the composition is an adult nutrition product.

8. The synthetic composition of claim 1, wherein the composition is an infant formula.

9. The synthetic composition of claim 1, further comprising one or more nondigestible oligosaccharides, preferably wherein the non-digestible oligosaccharide is selected from the group consisting of fructo-oligosaccharides and galacto-oligosaccharides.

10. The synthetic composition of claim 1, wherein at least 0.6 wt % of the fatty acid acyl groups present in the lipid fraction consists of 4 carbon atoms; and/or
wherein at least 10% of the fatty acid acyl groups present in the lipid fraction is a palmitoyl group (CH₃(CH₂)₁₄C(O)) and at least 30% of the palmitoyl groups is esterified to the sn-2 position of a triglyceride based on total palmitoyl groups.

11. The synthetic composition of claim 1, wherein at least 20 wt % of the lipid fraction consists of milk fat, preferably at least 30 wt %, more preferably at least 40 wt %.

12. The synthetic composition of claim 1, wherein the composition comprises at least 20 nanogram TGF β2 per ml; and/or at least 1*10⁻⁵ ng TGF β2 per gram of protein.

13. A method for the prevention of a virus infection of the respiratory tract comprising administering the synthetic composition of claim 1 to a subject in need thereof.

14. Use of a composition comprising TGF β2, in the prevention of a virus infection of the respiratory tract, wherein the composition is as defined in claim 1; wherein the use comprises the step of administering the composition comprising TGF β2 to a subject in need thereof.
